(19) United States
(12) Patent Application Publication
(10) Pub. No.: US 2022/0218857 A1
(43) Pub. Date: Jul. 14, 2022

(54) ROBOT FOR ATOMIZATION AND DISINFECTION

(71) Applicant: Shenzhen Institute of Geriatrics, Shenzhen (CN)

(72) Inventors: Zhengzhi Wu, Shenzhen (CN); Chunbao Wang, Shenzhen (CN); Quanquan Liu, Shenzhen (CN); Lihong Duan, Shenzhen (CN); Xin Zhang, Shenzhen (CN); Tong Wang, Shenzhen (CN); Zhixian Mao, Shenzhen (CN); Guangshuai Zhang, Shenzhen (CN); Chengkai Luo, Shenzhen (CN); Wanfeng Shang, Shenzhen (CN); Yajing Shen, Shenzhen (CN); Zhuohua Lin, Shenzhen (CN); Chunyu Wang, Shenzhen (CN); Riwei Zhang, Shenzhen (CN); Limin Li, Shenzhen (CN)

(21) Appl. No.: 17/564,682

(22) Filed: Dec. 29, 2021

(30) Foreign Application Priority Data
Jan. 12, 2021 (CN) 2021100367862

(51) Int. Cl. A61L 2/22; A61L 2/26; A61L 2/24; A61L 9/14; A61M 11/02; G05D 1/02; G01N 33/00

(52) U.S. Cl. CPC A61L 2/22; G05D 2201/0211; A61L 2/24; A61L 9/14; A61M 11/02; G05D 1/0248; G05D 1/0276; G01N 33/0073; A61L 2202/25; A61L 2202/16; A61L 2202/15; A61L 2202/14; A61L 2209/21; A61L 2209/111; A61M 2206/20; A61M 2205/3379; A61M 2205/3313; A61M 2205/3368; A61L 2/26

(57) ABSTRACT

A robot for atomization and disinfection is provided, which includes a robot body, an aerosol generator arranged outside the robot body for spraying aerosol mist particles into air, an aerosol concentration detector arranged outside the robot body for detecting an aerosol concentration in the air, and a main controller arranged inside the robot body and connected to the aerosol generator and the aerosol concentration detector for controlling a spray volume of the aerosol mist particles in real time according to the aerosol concentration after the aerosol generator sprays aerosol mist particles into the air. The aerosol concentration is dynamically monitored in real time, and the sterilization aerosol is supplemented in real time according to the concentration change, so as to maintain the stability of the concentration of the sterilization aerosol, improve the intelligence of space disinfection for epidemic prevention, and reduce the burden of manual disinfection.

### FIELD OF DISCLOSURE

The disclosure relates to the field of robotics, and more particularly to a control system and a robot for atomization and disinfection.

### BACKGROUND OF DISCLOSURE

Space disinfection for epidemic prevention is an important means to prevent the spread of virus. The conventional way of disinfection is mainly artificial spray, and the burden is heavy. Moreover, the concentration of aerosols in the air is difficult to meet requirements of the epidemic prevention due to space air and personnel flow.

### SUMMARY OF DISCLOSURE

The disclosure provides a robot for atomization and disinfection (also referred to as atomizing disinfection robot) to realize an intelligent maintenance of sterilization aerosol, improve an intelligence of space disinfection and epidemic prevention, and reduce a burden of manual disinfection.

Specifically, the embodiment of the disclosure provides a robot for atomization and disinfection, which may include a robot body, an aerosol generator and an aerosol concentration detector arranged outside the robot body, and a main controller arranged inside the robot body.

The aerosol generator is configured for spraying aerosol mist particles into air.

The aerosol concentration detector is configured for detecting an aerosol concentration in the air.

The main controller is connected to the aerosol generator and the aerosol concentration detector, and is configured to control a spray volume of the aerosol mist particles sprayed from the aerosol generator in real time according to the aerosol concentration in the air detected by the aerosol concentration detector after the aerosol generator sprays the aerosol mist particles into the air.

In an embodiment, the robot for atomization and disinfection may further include:

a communicator connected to the main controller and configured for feeding back at least one of the aerosol concentration in the air detected by the aerosol concentration detector and the spray volume of the aerosol mist particles sprayed by the aerosol generator to an external monitoring center, and receiving a disinfection control command from the external monitoring center. The disinfection control command is configured to control the spray volume of the aerosol mist particles sprayed by the aerosol generator.

In an embodiment, the robot for atomization and disinfection may further include:

an infrared camera connected to the main controller and configured for real-time monitoring body temperature information of surrounding mobile personnel.

The main controller is further configured to judge whether the monitored body temperature information of the surrounding mobile personnel is abnormal, and send warning information to the external monitoring center through the communicator when the monitored body temperature information of the surrounding mobile personnel is abnormal.

In an embodiment, the robot for atomization and disinfection may further include:

a navigation camera connected to the main controller and configured for acquiring environmental image information around the robot.

The main controller is further configured to control at least one motion control quantity of the robot according to the environmental image information, and the at least one motion control quantity may include one or more selected from a group consisting of motion speed, motion angle and motion distance.

In an embodiment, the communicator is further configured to receive a motion control command from the external monitoring center.

The main controller is further configured to control the motion control quantity of the robot according to the motion control command.

In an embodiment, the robot for atomization and disinfection may further include:

a laser rangefinder connected to the main controller and configured for obtaining current distance information of an obstacle around the robot in a preset direction.

The main controller is further configured to control the motion control quantity of the robot according to the current distance information.

In an embodiment, the aerosol generator is configured to use compressed air to pass through a fine atomization nozzle in form of a high-speed air flow to generate a negative pressure around the fine atomization nozzle and thereby carry a liquid medicine of a liquid storage tank into the high-speed air flow to thereby smash the liquid medicine into aerosol mist particles of different sizes. Droplets of large mist particles of the aerosol mist particles fall back into the liquid storage tank through a collision of a return baffle, and remaining small mist particles of the aerosol mist particles are sprayed out at a high speed to form aerosol-like medicine particles in the air.

In an embodiment, the aerosol concentration detector is configured to pump the air through an air pump into a detection chamber to form an air flow, irradiate the air flow by light emitted from a LED light source, detect an absorbed rate of the light by the aerosol mist particles in the air flow, and thereby determine the aerosol concentration in the air.

In an embodiment, the main controller is further configured to construct a real-time track planning map path of the robot according to the environmental image information and thereby control the robot to move along the real-time track planning map path.

In an embodiment, an application scenario of the robot for atomization and disinfection may include one or more selected from a group consisting of hospital, hotel, shopping mall and station.

Compared with the prior art, the embodiments of the disclosure may mainly have the following beneficial effects.

The aerosol concentration is dynamically monitored in real time, and the sterilization aerosol is supplemented in real time according to a concentration change, the stability of the sterilization aerosol concentration is maintained, the intelligence of space disinfection and epidemic prevention is improved, and the burden of manual disinfection is reduced.

### BRIEF DESCRIPTION OF DRAWINGS

FIG. 1 is a schematic block diagram of a robot for atomization and disinfection in embodiment 1 of the disclosure.

FIG. 2 is a schematic structural diagram of an aerosol generator in the embodiment 1 of the disclosure.

FIG. 3 is a schematic structural diagram of an aerosol concentration detector in the embodiment 1 of the disclosure.

FIG. 4 is a schematic block diagram of a robot for atomization and disinfection in embodiment 2 of the disclosure.

FIG. 5 is a schematic systematic diagram of an intelligent monitoring system of environment and body temperature in the embodiment 2 of the disclosure.

FIG. 6 is a schematic systematic diagram of autonomous navigation and remote-control navigation of the robot in the embodiment 2 of the disclosure.

FIG. 7 is a schematic systematic diagram of autonomous obstacle avoidance system of the robot in the embodiment 2 of the disclosure.

### DETAILED DESCRIPTION OF EMBODIMENTS

The disclosure will be further described in detail below in combination with the accompanying drawings and embodiments. It can be understood that the specific embodiments described herein are only used to explain the disclosure and not to limit the disclosure. In addition, it should be noted that for ease of description, only some but not all structures related to the disclosure are shown in the drawings.

In the description of the disclosure, it should be noted that the orientation or positional relationship indicated by the terms “center”, “up”, “down”, “left”, “right”, “vertical”, “horizontal”, “inside”, “outside” is based on the orientation or positional relationship shown in the accompanying drawings, which is only for the convenience of describing the disclosure and simplifying the description, rather than indicating or implying that the device or element must have a specific orientation, be configured and operated in a specific orientation, and therefore cannot be understood as a limitation of the disclosure. In addition, the terms “first” and “second” are used for descriptive purposes only and cannot be understood as indicating or implying relative importance. The terms “first position” and “second position” are two different positions.

In the description of the disclosure, it should be noted that the terms “installation”, “connect to” and “connection” should be understood in a broad sense unless otherwise clearly specified and limited, for example, they can be fixed connection or detachable connection; they can be mechanical connection or electrical connection; they can be connected directly or indirectly through an intermediate medium, and they can be a connection between the two elements. For those skilled in the related art, the specific meaning of the above terms in the disclosure can be understood in specific situations.

### Embodiment 1

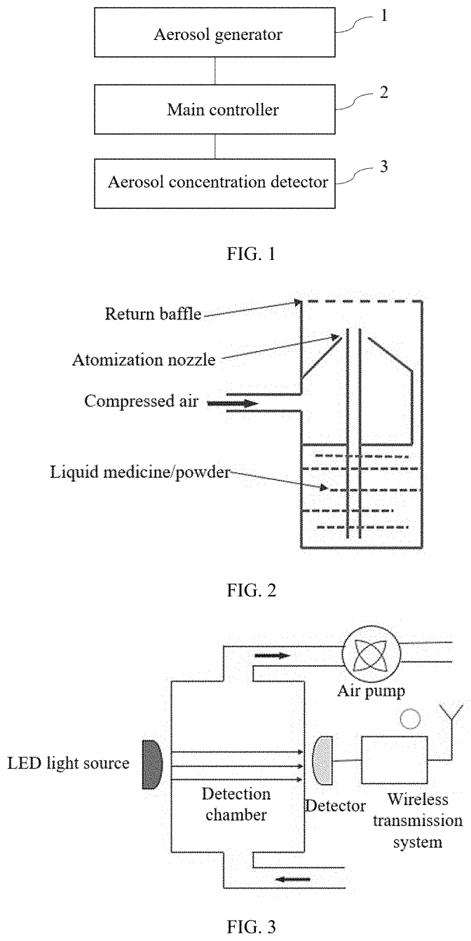

FIG. 1 is a schematic block diagram of a robot for atomization and disinfection provided by embodiment 1 of the disclosure. The illustrated embodiment of the disclosure can be applied to a disinfection of the robot. Referring to FIG. 1, the robot for atomization and disinfection according to the illustrated embodiment of the disclosure specifically includes a robot body, an aerosol generator 1 and an aerosol concentration detector 3 arranged outside the robot body, and a main controller 2 arranged inside the robot body.

The aerosol generator 1 is configured for spraying aerosol mist particles into air.

Specifically, the aerosol generator 1 realizes the quantitative output of aerosols according to physical characteristics of different medicines. As shown in FIG. 2, the aerosol generator uses compressed air to pass through a fine atomization nozzle in form of a high-speed air flow. According to the venturi effect, a negative pressure is generated around the nozzle and thereby carry a liquid medicine of a liquid storage tank into the high-speed air flow to smash the liquid medicine into aerosol mist particles of different sizes. The droplets of large mist particles fall back into the liquid storage tank through the collision of a return baffle, and the remaining small mist particles are sprayed at a high speed to from aerosol-like liquid medicine particles in the air. A particle spectrum diameter ranges from 5 micrometers (μm) to 100 μm (determined by different nozzles), the particle size is stable and can float in the air for a long time.

The aerosol concentration detector 3 is configured to detect an aerosol concentration in the air.

Specifically, the aerosol concentration detector 3 is designed based on a principle of spectral detection. According to different characteristics of different spectral absorbed rate of medicines, a target LED light source is selected to determine the aerosol concentration (also referred to as sterilization aerosol concentration) in the air by detecting the absorbed rate of the target light source. As shown in FIG. 3, the aerosol concentration detector 3 pumps the air through the air pump into a detection chamber to form an air flow. The LED light source is configured to irradiate the air flow by light, detect an absorbed rate of light absorbed by the aerosol mist particles in the air flow, and determine the sterilization aerosol concentration in the air.

The main controller 2 is connected to the aerosol generator 1 and the aerosol concentration detector 3 to control a spray volume of aerosol mist particles from the aerosol generator 1 in real time according to an aerosol concentration in the air detected by the aerosol concentration detector 3 after the aerosol generator 1 sprays aerosol mist particles into the air.

Specifically, the main controller 2 controls the spray volume of aerosol mist particles from the aerosol generator 1 according to a preset required aerosol concentration. At the same time, the aerosol concentration detector 3 detects the aerosol concentration in the air and feeds it back to the main controller 2. The main controller 2 adjusts the spray volume of aerosol mist particles from the aerosol generator 1 in real time according to the concentration. When the aerosol concentration in the current air is too low, the main controller 2 controls the aerosol generator 1 to increase the spray volume of sprayed aerosol mist particles. When the aerosol concentration in the current air is too high, the main controller 2 controls the aerosol generator 1 to reduce the spray volume of sprayed aerosol mist particles and maintain the stability of the sterilization aerosol concentration in the air.

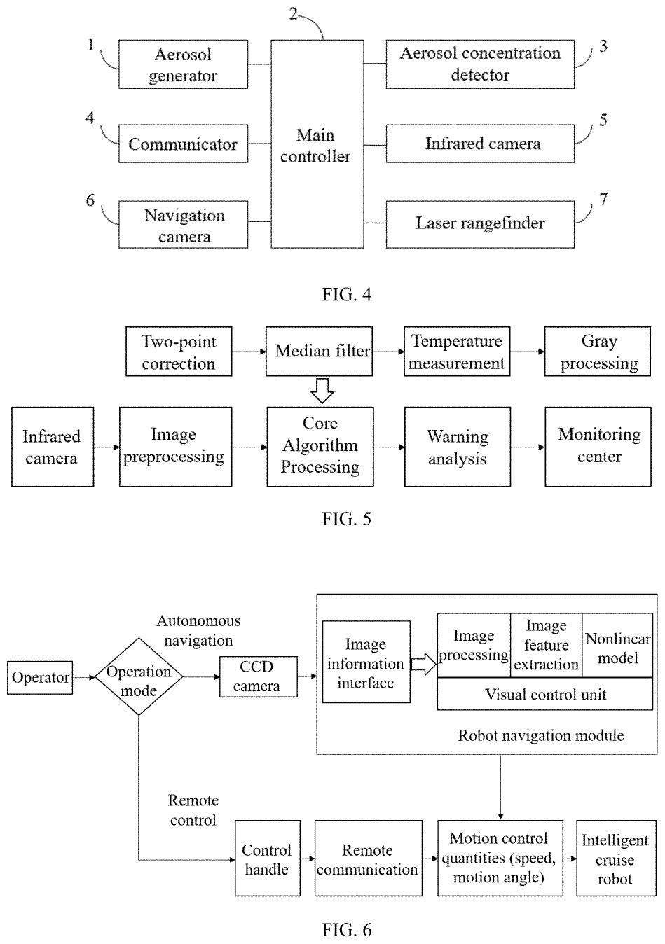

In an illustrated embodiment, as shown in FIG. 4, the robot for atomization and disinfection further includes a communicator 4 (also referred to as a communication module, such as WiFi module, Bluetooth module, mobile communication module, or other wireless communication module) connected to the main controller 2, which is configured to feed back at least one of the aerosol concentration in the air detected by the aerosol concentration detector 3 and the spray volume of aerosol mist particles sprayed by the aerosol generator 1 to the external monitoring center, and receive a disinfection control command from the external monitoring center. The disinfection control command is used to control the spray volume of aerosol mist particles sprayed by aerosol generator 1. It can be understood that the communication module (communicator) includes a processor and a memory connected to the processor, and the memory includes software modules, executable by the processor.

Specifically, the communicator 4 can send the aerosol concentration in the air detected by the aerosol concentration detector 3 and/or the spray volume of aerosol mist particles sprayed by the aerosol generator 1 to the monitoring center in real time. For example, the aerosol concentration in the air and/or the spray volume of aerosol mist particles can be displayed on a large screen of the monitoring center, so that the staff can understand the current disinfection situation of the robot for atomization and disinfection in real time. Moreover, the monitoring center can also send a disinfection control command to the robot to command the robot to work according to the instructions of the staff, for example, to control the spray volume of aerosol mist particles from aerosol generator 1.

In the technical solution of the embodiment of the disclosure, the aerosol concentration is dynamically monitored in real time, and the sterilization aerosol is supplemented in real time according to the concentration change, so as to maintain the stability of the concentration of the sterilization aerosol, improve the intelligence of space disinfection and epidemic prevention, and reduce the burden of manual disinfection.

### Embodiment 2

FIG. 4 is a schematic block diagram of a robot for atomization and disinfection provided by embodiment 2 of the disclosure. Referring to FIG. 4, on the basis of the embodiment 1, the robot for atomization and disinfection of this illustrated embodiment may further include an infrared camera 5, a navigation camera 6 and a laser rangefinder 7.

The infrared camera 5 is connected to the main controller 2 and configured for real-time monitoring body temperature information of surrounding mobile personnel. The main controller 2 is configured to judge whether the body temperature information of the surrounding mobile personnel is abnormal. The main controller 2 will send warning information to the monitoring center through the communicator 4 when the body temperature information of the surrounding mobile personnel is abnormal.

Specifically, as shown in FIG. 5, by installing the infrared camera on the robot, the body temperature of the surrounding mobile personnel is monitored in real time. After image preprocessing, the body temperature of the personnel in the environment is calculated through core algorithm processing, including two-point correction, median filtering, temperature measurement, gray processing, etc, and the gray information and the warning information shall be sent to the monitoring center to feed back the temperature collection data in time. If there is any abnormality in the temperature information of personnel, warning shall be given. In the illustrated embodiment, the core algorithm processing adopts the existing algorithm, and other algorithms can also be adopted, which is not limited in this embodiment.

The navigation camera 6 is connected to the main controller 2 and configured for acquiring environmental image information around the robot. The main controller 2 is configured to control at least one motion control quantity of the robot according to the environmental image information. The at least one motion control quantity includes one or more selected from a group consisting of motion speed, motion angle and motion distance. The communicator 4 is further configured to receive a motion control command from the monitoring center. The main controller 2 is further configured to control the motion control quantity of the robot according to the motion control command.

Specifically, because the working environment of the robot for atomization and disinfection is complex and uncertain, such as pedestrians passing by, the robot is required to recognize the surrounding environment in real time and respond in time. Therefore, it is necessary to build a multi-level information sensing system and develop an intelligent cruise robot system through multi-sensor information fusion technology. As shown in FIG. 6, in the face of an unknown working environment, the remote and autonomous navigation of the robot for atomization and disinfection are realized by using the navigation camera 6 and the machine vision technology. Combined with the robot vision and intelligent control algorithm, the nonlinear mapping of the robot navigation module from the visual image information to the robot motion control quantity is completed, so as to realize the robot path navigation. The motion control quantity includes but is not limited to motion speed, motion angle, motion distance, etc. It can be understood that the robot navigation module includes a processor and a memory connected to the processor, and the memory includes software modules, executable by the processor.

In an illustrated embodiment, the main controller 2 is further configured to construct a real-time track planning map path of the robot according to the environmental image information, and control the robot to move along the path.

Specifically, the operator decides operation mode (i.e., working mode) of the cruise robot in advance. In a remote-control mode, the operator obtains the on-site environment image of the robot from the navigation camera 6 on the robot platform, and then controls the moving direction and speed of the robot through the handle. In an autonomous cruise mode of the robot, the road condition information is obtained simultaneously through the RGB camera and the depth camera, so as to obtain the depth information while obtaining the image information of the road surface and obstacles, and transmit it to the main controller 2 through the image interface. The main controller 2 carries out image processing and image feature extraction to obtain effective road condition information. The visual control core unit uses intelligent algorithm to map the effective road condition information to the vehicle driving motion control quantity. It can be understood that the visual control core unit includes a processor and a memory connected to the processor, and the memory includes software modules, executable by the processor.

The laser rangefinder 7 (also referred to as laser ranging unit) is connected to the main controller 2 and configured for obtaining a current distance information of an obstacle around the robot in a preset direction. The main controller 2 is configured to control the motion control quantity of the robot according to the current distance information.

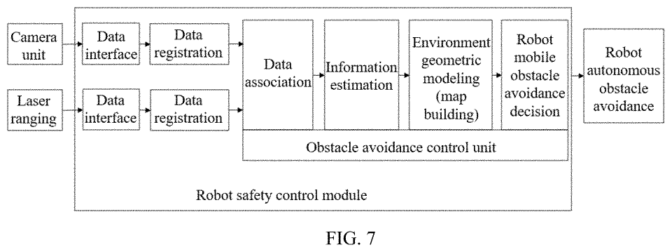

Specifically, as shown in FIG. 7, a camera unit and a laser lidar are installed on the top of the robot to build a 360-degree continuous rotation support mechanism. During the working process of the robot, the camera unit and laser lidar continuously monitor the surrounding environment. Through the difference of detection distance and accuracy of the sensor unit, a detection mode of distance matching, rapid movement and accurate movement in complex environment is formed, and a closed loop of robot trajectory control is constructed to control the safe movement of the robot.

In an illustrated embodiment, an application scenario of the robot for atomization and disinfection includes but are not limited to hospital, hotel, shopping mall, station, etc.

In the technical solution of the illustrated embodiment of the disclosure, the aerosol concentration is dynamically monitored in real time, and the sterilization aerosol is supplemented in real time according to the concentration change, so as to maintain the stability of the concentration of the sterilization aerosol, improve the intelligence of space disinfection and epidemic prevention, and reduce the burden of manual disinfection.

It is noted that the above is only the illustrated embodiments of the disclosure and the applied technical principle. Those skilled in the related art will understand that the disclosure is not limited to the specific embodiments described herein, and various obvious changes, readjustments and substitutions can be made for those skilled in the related art without departing from the protection scope of the disclosure. Therefore, although the disclosure has been described in more detail through the above embodiments, the disclosure is not limited to the above embodiments, but can also include more other equivalent embodiments without departing from the concept of the disclosure, and the scope of the disclosure is determined by the scope of the appended claims.

What is claimed is:

1. A robot for atomization and disinfection, comprising:
a robot body;
an aerosol generator, arranged outside the robot body and configured for spraying aerosol mist particles into air,
an aerosol concentration detector, arranged outside the robot body and configured for detecting an aerosol concentration in the air; and
a main controller, arranged inside the robot body and connected to the aerosol generator and the aerosol concentration detector, wherein the main controller is configured to control a spray volume of the aerosol mist particles sprayed from the aerosol generator in real time according to the aerosol concentration in the air detected by the aerosol concentration detector after the aerosol generator sprays the aerosol mist particles into the air.

2. The robot according to claim 1, further comprising:
a communicator, connected to the main controller;
wherein the communicator is configured for feeding back at least one of the aerosol concentration in the air detected by the aerosol concentration detector and the spray volume of the aerosol mist particles sprayed by the aerosol generator to an external monitoring center, and receiving a disinfection control command from the external monitoring center; and the disinfection control command is configured to control the spray volume of the aerosol mist particles sprayed by the aerosol generator.

3. The robot according to claim 2, further comprising:
an infrared camera, connected to the main controller and configured for real-time monitoring body temperature information of surrounding mobile personnel;
wherein the main controller is further configured to judge whether the monitored body temperature information of the surrounding mobile personnel is abnormal, and send warning information to the external monitoring center through the communicator when the monitored body temperature information of the surrounding mobile personnel is abnormal.

4. The robot according to claim 2, further comprising:
a navigation camera, connected to the main controller and configured for acquiring environmental image information around the robot;
wherein the main controller is further configured to control at least one motion control quantity of the robot according to the environmental image information, and the at least one motion control quantity comprises one or more selected from a group consisting of motion speed, motion angle and motion distance.

5. The robot according to claim 4, wherein the communicator is further configured to receive a motion control command from the external monitoring center; and
wherein the main controller is further configured to control the motion control quantity of the robot according to the motion control command.

6. The robot according to claim 5, further comprising:
a laser rangefinder, connected to the main controller and configured for obtaining current distance information of an obstacle around the robot in a preset direction;
wherein the main controller is further configured to control the motion control quantity of the robot according to the current distance information.

7. The robot according to claim 1, wherein the aerosol generator is configured to use compressed air to pass through an atomization nozzle in form of an air flow to generate a negative pressure around the atomization nozzle and thereby carry a liquid medicine of a liquid storage tank into the air flow to smash the liquid medicine into aerosol mist particles of different sizes;
wherein droplets of large mist particles of the aerosol mist particles fall back into the liquid storage tank through a collision of a return baffle, remaining small mist particles of the aerosol mist particles are sprayed out to form aerosol-like liquid medicine particles in the air.

8. The robot according to claim 1, wherein the aerosol concentration detector is configured to pump the air through an air pump into a detection chamber to form an air flow, irradiate the air flow by light emitted from a LED light source, detect an absorbed rate of the light by the aerosol mist particles in the air flow, and thereby determine the aerosol concentration in the air.

9. The robot according to claim 4, wherein the main controller is further configured to construct a real-time track planning map path of the robot according to the environmental image information and thereby control the robot to move along the real-time track planning map path.

10. The robot according to claim 1, wherein an application scenario of the robot for atomization and disinfection comprises one or more selected from a group consisting of hospital, hotel, shopping mall and station.
